(19) United States
(12) Patent Application Publication
(10) Pub. No.: US 2005/0288280 A1
(43) Pub. Date: Dec. 29, 2005

(54) Meloxicam in veterinary medicine

(75) Inventors: Gabriele Friton, Heubach (DE); Ernst Salamon, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Vetmedica GmbH, Ingelheim (DE)

(21) Appl. No.: 11/127,990

(22) Filed: May 12, 2005

(30) Foreign Application Priority Data
Jun. 23, 2004 (DE) 10 2004 030 409

(51) Int. Cl. A61K 31/5415

(52) U.S. Cl. 514/226.5

(57) ABSTRACT

A veterinary pharmaceutical formulation containing meloxicam or a pharmacologically acceptable meloxicam salt of an organic or inorganic base and one or more vehicles having analgesic efficacy for the treatment of inflammatory painful diseases, particularly for the treatment of mild or moderate mastitis cases. The treatment leads to an effective long lasting reduction of a hypersensitive state associated with inflammatory pain in mild or moderate mastitis cases, particularly chronic states thereof.

### RELATED APPLICATIONS

This application claims priority to German Patent Application No. 10 2004 030 409.2, filed on Jun. 23, 2004, which application is incorporated herein in its entirety.

### FIELD OF THE INVENTION

The present invention is directed to the novel use of meloxicam in veterinary medicine, especially for the treatment of painful conditions in mild and moderate bovine mastitis cases.

### BACKGROUND OF THE INVENTION

Mastitis is one of the most important diseases in dairy cattle and a serious cause of loss to the world's dairy industries. The U.S. National Mastitis Council (NMC) estimates that annual losses to the dairy industry amount to US $1.8 to 2 billion or US $185 to 200 per cow. Losses due to discarded milk alone are thought to amount to US $1 billion. Up to 50% of all dairy cattle are thought to be affected by mastitis. Besides significant economic losses, mastitis affects cow welfare.

Mastitis is an inflammation of the mammary gland and results in significant losses to dairy industry due to production decrease or increased culling rates. Mastitis-causing pathogens can be divided into contagious pathogens that are associated with the udder (i.e., Staphylococcus aureus, Streptococcus agalactiae, and Streptococcus dysgalactiae) and environmental pathogens that are present in a cow's environment (coliform bacteria and streptococci other than Streptococcus agalactiae, and coagulase-negative staphylococci).

Infection may be clinically obvious with severe signs of illness in acute mastitis cases or depending on chronic clinical or subclinical mastitis cases associated with mild up to moderate general signs and/or mild up to obvious local inflammatory signs. General clinical signs refer to increased rectal temperature (up to high fever) and severe deterioration in a cow's general state of health (no or reduced feed intake, impaired general condition). Local clinical signs include changes in macroscopic milk quality associated with typical inflammatory reactions of the affected quarter (red, swollen, warm, and painful). Both acute and chronic mastitis lead to reduced milk production. Additionally, in acute mastitis cases with severe local inflammatory signs it is well recognized that this disease affects animal's performance and wellbeing.

Most important for milk production loss is chronic mastitis due to the chronic and irreversible tissue damage. The extent of udder infection in a dairy cow is usually assessed through measuring the number of somatic cells present in milk.

Recognition, alleviation, and control of pain and stress are central to ensuring good welfare in food producing animals. Conditions such as mastitis and lameness in cows are highly prevalent and of significant welfare concern. Inflammation induces alterations in nociceptive information processing which may have serious consequences for the animal.

Allodynia (perception of innocuous stimuli as noxious) and hyperalgesia (exaggerated response to noxious stimuli) are the common denominators of inflammatory pain. The duration of this hypersensitized state may long outlast the inflammatory stimulus and resolution of clinical signs, which has serious implications for welfare.

Work in cattle with acute lameness has documented hyperalgesia (H. R. Whay, A. E. Waterman, A. J. Webster, and J. K. O'Brien, 1998, The influence of lesion type on the duration of hyperalgesia associated with hind limb lameness in dairy cattle, Vet J. 1998 July; 156(1), P 23-29), which outlasted clinical resolution of the disease, while preliminary studies in dairy cows with acute mastitis (mild or moderate) indicated that abnormal pain processing was present for up to 40 days (Fitzpatrick and Nolan, unpublished observations). Mastitis is an inflammatory disease likely to induce pain; indeed bradykinin, a hyperalgesic mediator, has been detected in milk from cases of clinical and subclinical mastitis in cows (H. R. Eshraghi, I. J. Zeitlin, J. L. Fitzpatrick, H. Ternent, and D. Logue, The release of bradykinin in bovine mastitis, Life Sci 1999; 64(18), P.1675-1687).

Antibiotics are commonly used for treatment and prevention of infections of the udder and various products are available. Antibiotics are usually administered by intramammary injection and in severe cases of infection, they may be administered parenterally in addition. Intramammary preparations are supplied in disposable single-use syringes or tubes.

It is further known that inflammation induces alterations in normal pain information processing, which may have serious consequences for the animal and which may be measured as hyperalgesia: an exaggerated response to noxious stimuli. In order to reduce local inflammatory conditions (anti-inflammatory including reduction of swelling, redness, heat, pain, and loss of function), steroidal anti-inflammatory drugs (SAID) are well established in being used in combination with antibiotics intramammarily. Furthermore, SAIDs can be administered systemically in combination with parenteral and/or intramammary antibiotic therapy.

Recently, the value of NSAIDs (non-steroidal anti-inflammatory drugs) in mastitis therapy, particularly in acute clinical mastitis cases, became of great importance, because NSAIDs have no immunosuppressive effect in comparison to SAIDs and they show proven efficacy in reduction of inflammation (anti-inflammatory), pain (analgesic), body temperature (anti-pyretic), and endotoxin associated clinical signs.

Currently there are several NSAIDs commercially available for cattle and licensed in several countries within the EU for being used in acute mastitis cases, i.e., flunixin meglumine (Finadyne® Injection, sold by Schering-Plough Animal Health), ketoprofen (Ketofen® 10%, sold by Merial), meloxicam (Metacam®, sold by Boehringer Ingelheim) and tolfenamid acid (Tolfedine®, Vetoquinol®). All products are licensed for parenteral administration; in some countries they are licensed for being used in dairy cows for the indication “as adjunctive therapy to antibiotics in acute clinical mastitis”.

NSAIDs are well recognized for relief of pain in the acute outbreak of diseases, however, their value in chronic states of diseases has not been evaluated for food producing animals. NSAIDs up to now known in prior art are not licensed for the treatment of painful conditions present in mild and moderate mastitis cases. For example, the NSAID flunixin meglumine (Finadyne® Injection, Schering-Plough Animal Health) did not achieve a long lasting analgesic activity when administered intramammarily in dairy cows (Fitzpatrick et al., Recognizing and controlling pain and inflammation in mastitis, Proc. British Mastitis Conference 1998, pp. 3444).

Currently there is no NSAID available on the market for the treatment of moderate up to mild mastitis cases with slightly increased rectal temperature as single general sign of illness and obvious up to mild local inflammatory signs, i.e., slight swelling of the gland and changed milk quality with either clots in milk and/or increased somatic cell counts.

Furthermore, the known prior art directed to the pharmaceutical use or the properties of the known NSAID meloxicam (Metacam®) is related to different aspects:

EP-A-0 002 482 shows, inter alia, the example of a 2.0% injectable solution of meloxicam consisting of the meglumine salt of the active substance, sodium chloride, and water.

EP-A-0 945 134 discloses the pH-dependent solubility characteristics of meloxicam and its salts, i.e., the sodium salt, the ammonium salt, and the meglumine salt, in aqueous solution.

WO 99/59634 A1 describes an eye drop solution containing 0.5% meloxicam.

Further, a commercially available 0.5% meloxicam solution is used in small animals such as dogs, heifers, and calves, for example, to treat respiratory diseases and inflammation.

WO 03/049733 describes a highly concentrated stable meloxicam solution for needleless injection containing from 35 to 100 mg/mL of dissolved meloxicam salt and one or more suitable additives for treating respiratory diseases and inflammation in mammals.

Finally, WO 01/97813 A2 describes aqueous cyclodextrin-free solutions of meloxicam for parenteral or oral administration which contain a pharmacologically acceptable meloxicam salt of an organic or inorganic base and one or more excipients characterized in that the content of dissolved meloxicam salt is more than 10 mg/mL.

Although the meloxicam formulations described in WO 03/049733 and WO 01/97813 A2 should be used in a pharmaceutical composition for treating pain, besides treating inflammation, fever, and respiratory complaints in large farm animals, such an efficacy to relief pain is clearly limited to acute mastitis cases. Furthermore, at that time, it was not possible to determine whether animals do feel pain in mastitis cases or if a pharmaceutical composition has an efficacy to alleviate the pain of the animals. A possibility for the recognition and assessment of pain in mastitis in dairy cows has been found recently. Therefore, a novel improved method is now available to determine if the animals feel pain (M. H. Milne, A. M. Nolan, P. J. Cripps, and J. L. Fitzpatrick, Preliminary results of a study on pain assessment in clinical mastitis in dairy cows, Proc. British Mastitis Conference (2003) Garstang, pp. 117-119).

Such method for the assessment of painful conditions, i.e., inflammatory chronic pain in food producing animals was developed and validated by using a mechanical device system. The method was established in dairy cows suffering from mild and moderate mastitis cases and validated. Surprisingly, cows suffering from mild to moderate mastitis cases suffer from painful conditions, which need, under animal welfare aspects, to be prevented and treated.

Therefore, it is an object of the present invention to provide a pharmaceutical composition for the treatment of inflammatory painful diseases, particularly mild and moderate mastitis cases, especially chronic states of diseases. The composition should also allow a treatment to be conducted systemically or locally as adjunct to antibiotics.

### DESCRIPTION OF THE INVENTION

Surprisingly, it has been found that a meloxicam formulation may be used for the treatment of painful conditions in animals. Therefore, the present invention provides the use of a formulation containing meloxicam or a pharmacologically acceptable meloxicam salt of an organic or inorganic base, one or more vehicles, and optionally one or more suitable additives, for preparing a veterinary medical composition having analgesic efficacy for the treatment of an inflammatory painful disease, particularly mild and/or moderate mastitis cases, especially chronic states thereof, in order to ameliorate hypersensitive states/inflammatory hyperalgesia related to local (chronic) inflammatory pain in the udder and particularly to relief pain.

Until now, the use of the meloxicam containing formulation according to the present invention has not been described for the application in mild and moderate mastitis cases but especially for acute mastitis cases, however, about 70% of the mastitis cases are chronic.

The recognition, alleviation and control of pain have been found to be important to ensure good welfare in animals. Therefore, as already mentioned, dairy cows were studied to assess pain associated with moderate or mild mastitis. Based on the abovementioned study directed to the newly established method for the measurement of pain in food producing animals, a clinical field study was used to investigate, besides painful chronic conditions in cows with mild and moderate mastitis, the analgesic efficacy and long duration of action of meloxicam. In such clinical field study, meloxicam was administered only once as adjunct to intramammary antibiotic therapy and was compared to a second group where animals received re-treatments with meloxicam after 3 and 6 days. A third group received the antibiotic stand alone therapy.

Surprisingly, the analgesic efficacy of a single administration of meloxicam showed comparable results to multiple administration of meloxicam. Therefore, the clinical field study including cows suffering from mild up to moderate mastitis cases, confirmed the analgesic efficacy of meloxicam with the new established and validated method for pain assessment and the results indicated that a single treatment with meloxicam achieved a long lasting analgesic efficacy. On the contrary, the antibiotic stand alone therapy did not ameliorate the painful condition of animals showing a significant difference to meloxicam treatment groups.

The analgesic effect was observed to occur very quickly and a single administration has a long lasting effect over several days resulting in a tremendous improvement of the wellbeing of the ill animals.

These investigations were conducted the first time in this indication area using meloxicam. Although a few prior art documents mention the treatment of pain with meloxicam formulations, a teaching which was not feasible or reproducible, the now developed method allows at the first time to determine and evaluate such an effect.

The other licensed NSAIDs which are parenterally administered are less likely to be used in chronic mild and moderate mastitis cases due to their either weak analgesic efficacy and/or due to the short duration of action (under 12 hours).

The latter would require additional re-treatments in short time intervals which is unlikely to be attractive to be used in food producing animals and the pharmaco-economic benefit for drug use would not be justified. In contrast, meloxicam offers unique benefits due to its long lasting duration of activity in cattle. The use of the meloxicam formulation of the invention in cows with mild and moderate mastitis cases leads to a decrease of the local inflammation symptoms (including a decrease of somatic cell counts in milk), a clinical improvement, as well as a reduction of the concentration of the inflammatory mediators and long lasting pain relief, which contributes to animal welfare.

In the frame of the present invention, the expression “mild and moderate mastitis cases” corresponds to differing grades of mastitis severity. Mild and moderate mastitis disease shows an alleviated course of disease compared with the acute mastitis and should be understood in the sense that the mastitis infection is either clinically obvious with less severe signs of illness than in acute mastitis cases or rather subclinically with practically no or hardly obvious clinical signs. If such a subclinical mastitis is not treated, a chronic mastitis will occur by-and-by having a clinical picture which is milder than an acute mastitis.

A rough rule of thumb is that average heart rates, respiratory rates, and rectal temperatures are higher in cows with moderate mastitis compared to cows with mild mastitis and compared to normal cows. According to the abovementioned studies (prior art study: M. H. Milne et al., Preliminary results of a study on pain assessment in clinical mastitis in dairy cows, ibid., and clinical field study of the present invention) mastitis is further classed as “mild” when there are changes in milk appearance, i.e., increase in somatic cell count with or without flakes or clots, but the udder is normal, and “moderate” when there are changes in milk appearance and the udder is hot, swollen, or painful to touch, but the cows are not “unwell” and/or no systemic antibiotic therapy is required. The elaborated method of the study on pain assessment also allows to assign borderline cases of the mastitis disease symptoms related with average heart rates, respiratory rates, and rectal temperatures either to moderate or mild mastitis (poster to the above paper presented at the British Mastitis Conference (2003)). Such definitions shall also apply for the present invention.

Therefore, the results of the clinical study according to the present invention indicates that meloxicam is beneficial in analgesic therapy of mild or moderate mastitis in dairy cows, the treatment with meloxicam has a significant effect and a single treatment with meloxicam achieves a long lasting analgesic efficacy.

The formulation according to the present invention may contain meloxicam or meloxicam salt in a concentration of 10-30 mg/mL, preferably 12-25 mg/mL, more preferably 16-23 mg/mL, particularly preferably 18-22 mg/mL, especially 20 mg/mL. It is particularly preferred if the content of dissolved meloxicam salt is less than 35 mg/mL.

The meloxicam-containing formulation used in the present invention may contain, in addition to meloxicam or a meloxicam salt, one or more vehicles and optionally one or more additives. Depending on the vehicle, the other additives are selected accordingly. The vehicle may be selected from water and/or oil to result in an aqueous or oily system; intermediate systems are also possible. The term “aqueous system” or “oily system” according to the present invention should be understood that the main part of the vehicle is derived from water or oil. The “vehicle” should be understood to be the medium or carrier which essentially disperses the active substance, i.e., the meloxicam or salt thereof, and the additives, if present, such that a formulation is formed.

The formulation used according to the present invention may be a liquid system such as an aqueous solution, a hydrogel, an emulsion such as a microemulsion, an oil-in-water emulsion, or a water-in-oil emulsion, or a suspension or the like. In the frame of the present invention the expression “solution” should be understood to comprise dispersed systems as well as true solutions and intermediate states. The formulation may further be a semi-solid or semi-liquid system such as a cream or an ointment, or a gaseous system such as a spray.

If an aqueous system is selected, the meloxicam is preferably used in the form of a salt. The meloxicam salt used according to the present invention may be the meglumine, sodium, potassium, or ammonium salt, preferably may be mentioned the meloxicam meglumine salt.

The meglumine concentration may be from 12.5 to 16.5 mg/mL, preferably 13-16 mg/mL, more preferably 13.5-15.5 mg/mL, most preferably 14-15 mg/mL, especially about 14 mg/mL. The possible sodium, potassium, and ammonium concentrations are calculated accordingly.

Meglumine and meloxicam may be used in a molar ratio of from 9:8 to 12:8, preferably in a molar ratio of 11:8, but especially in a molar ratio of 10:8.

The additives used may be any of those which are permitted under the drug licensing laws and known from the prior art, but exemplary mentioned additives may be buffers, solubilizers, gelling agents, viscosity enhancers, preservatives, oils, antioxidants, emulsifiers, foam forming agents, isotonic agents, a propellant gas, and/or thickeners.

Other suitable additives are, for example, citric acid, lecithin, gluconic acid, tartaric acid, phosphoric acid, and EDTA or the alkali metal salts thereof, preferably tartaric acid and EDTA or the alkali metal salts thereof, particularly disodium EDTA.

In an aqueous medium it is advantageous if the additives are selected from the group consisting of small concentrations of solubilizers, a preservative, a buffer substance for achieving the optimum pH range, and optionally other additives. The system may, for example, optionally contain a suitable gelling agent and/or viscosity enhancer leading to a viscous aqueous solution or a hydrogel. Suitable systems can be sterile viscous aqueous solutions or hydrogels, sterile emulsions (e.g., oil-in-water), or sterile oily suspensions.

If an oily system is selected the additives may preferably be selected from one or more oils, one or more antioxidants, and optionally one or more thickeners. It is a matter of course that also other additives may be present.

The additives being present in the formulation will be described hereinafter in detail.

As solubilizers may be used any known solubilizer suitable in the veterinary medical sector, for example, polyethyleneglycols, polyoxyethylene-polyoxypropylene copolymers (e.g., poloxamer 188), glycofurol, arginine, lysine, castor oil, propyleneglycol, solketal, polysorbate, glycerol, sorbitol, mannitol, xylitol, polyvinylpyrrolidone, lecithin, cholesterol, 12-hydroxystearic acid-PEG660-ester, propyleneglycol monostearate, polyoxy-40-hydrogenated castor oil, polyoxyl-10-oleyl-ether, polyoxyl-20-cetostearylether, and polyoxyl-40-stearate or a mixture of sorbitol, mannitol, and xylitol. Preferred are polyethyleneglycols, polyoxyethylene-polyoxypropylene copolymers, glycofurol, polyvinylpyrrolidone, lecithin, cholesterol, 12-hydroxystearic acid-PEG660-esters, propyleneglycol monostearate, polyoxy-40-hydrogenated castor oil, polyoxyl-10-oleyl-ether, polyoxyl-20-cetostearylether, and polyoxyl-40-stearate. Particularly preferred are polyethyleneglycols, glycofurol, and polyoxyethylene-polyoxypropylene-copolymers, but especially polyethyleneglycols (e.g., Macrogol 300) and polyoxyethylene-polyoxypropylene copolymers (e.g., Poloxamer 188).

The concentration of the solubilizers may be in the range from 20-200 mg/mL, preferably 30-150 mg/mL, more preferably 40-130 mg/mL, most preferably 50-120 mg/mL, and especially 70-100 mg/mL.

Any preservatives known for use in the pharmaceutical field may be used, for example, ethanol, benzoic acid and the sodium or potassium salts thereof, sorbic acid and the sodium or potassium salts thereof, chlorobutanol, benzyl alcohol, phenylethanol, phenylmercury nitrate, methyl, ethyl, propyl, or butyl-p-hydroxybenzoates, phenol, m-cresol, p-chloro-m-cresol or benzalkonium chloride. Preferred are ethanol, benzoic acid and the sodium or potassium salts thereof, sorbic acid and the sodium or potassium salts thereof, chlorobutanol, benzyl alcohol, phenylethanol, and methyl, ethyl, propyl or butyl p-hydroxybenzoates, but particularly preferred are ethanol, benzoic acid and the sodium or potassium salts thereof, and sorbic acid and the sodium or potassium salts thereof, but especially ethanol.

The concentration of the preservative ethanol may be in the range from 100-200 mg/mL, preferably 120-180 mg/mL, and more preferably about 150 mg/mL.

The concentration of the preservatives benzoic acid and the sodium or potassium salts thereof, sorbic acid and the sodium or potassium salts thereof, chlorobutanol, benzyl alcohol, phenylethanol, phenol, m-cresol, and p-chloro-m-cresol may be in the range from 0.5-50 mg/mL, preferably 1-10 mg/mL, and more preferably 3-5 mg/mL.

The concentration of the preservatives benzalkonium chloride, phenylmercury nitrate, and methyl, ethyl, propyl, or butyl-p-hydroxybenozates may be in the range from 0.01-4 mg/mL, preferably 0.02-3 mg/mL, and more preferably 0.1-0.5 mg/mL.

It may be advantageous if the formulation containing an aqueous medium according to the invention has a pH value in the alkaline range. Then, the pH value may be adjusted in the range from about 8 to about 10, preferably from about 8.5 to about 9, more preferably a pH from about 8.7 to about 8.9, particularly about 8.8. However, also a pH value in the acidic range may be possible but an alkaline pH range is particularly preferred. In the more alkaline region the meloxicam containing formulation tends preferably to be a true aqueous solution whereas in the more acidic region it tends rather to be a suspension. Therefore, the buffer system used to achieve a pH value of from about 8 to about 10 may be, for example, glycine, a mixture of glycine and HCl, a mixture of glycine and sodium hydroxide solution, and the sodium and potassium salts thereof, a mixture of potassium hydrogen phthalate and hydrochloric acid, a mixture of potassium hydrogen phthalate and sodium hydroxide solution, or a mixture of glutamic acid and glutamate. Glycine, a mixture of glycine and HCL and a mixture of glycine/sodium hydroxide solution, especially glycine, are particularly preferred.

The concentration of the buffer substances may be from 4 to 50 mg/mL, preferably from 5 to 20 mg/mL, and more preferably from 8 to 10 mg/mL. The concentration of the other additives mentioned above, i.e., EDTA, citric acid, lecithin, gluconic acid, tartaric acid, and phosphoric acid or the salts thereof may be in the range from 0.2-3 mg/mL, preferably 0.3-2.5 mg/mL, more preferably 0.5-2 mg/mL, most preferably 0.6-1.5 mg/mL, and in particular 0.7-1.0 mg/mL.

One preferred formulation of the invention contains, in addition to the meglumine or sodium salt of the meloxicam, polyethyleneglycols, glycofurol and/or polyoxyethylene-polyoxypropylene copolymers, but particularly polyethyleneglycols (e.g., Macrogol 300) and/or polyoxyethylene-polyoxypropylene copolymers (e.g., Poloxamer 188) as solubilizer, ethanol, benzoic acid and the sodium or potassium salts thereof, or sorbic acid and the sodium or potassium salts thereof, but particularly ethanol, as preservative, and glycine, a mixture of glycine/HCl or a mixture of glycine/sodium hydroxide solution, but preferably glycine, as buffer and optionally disodium EDTA as an additional additive.

In the formulation according to the invention, meloxicam and the other additives, particularly disodium EDTA, may be present in a weight ratio of from 25:1 to 15:1, preferably from 24:1 to 16:1, preferably from 23:1 to 17:1, more preferably from 22:1 to 18:1, and most preferably from 21:1 to 19:1, in particular about 20:1.

In the oily system suitable oily components are any of the active substances known from the prior art for the preparation of pharmaceuticals, such as, for example, vegetable oils, in particular, e.g., cotton seed oil, groundnut oil, maize oil, rapeseed oil, sesame oil, and soya oil, or triglycerides of moderate chain length, e.g., fractionated coconut oil, or isopropylmyristate, -palmitate, or mineral oils or ethyloleate or mixtures thereof. Preferred oils may be selected from vegetable oils, such as corn seed oil, sesame oil, and peanut oil.

The antioxidants used in oily systems may be any of the antioxidants known from the prior art, preferably sesamol, alpha-tocopherol (vitamin E), butylhydroxytoluene (BHT), or butylhydroxyanisole (BELA).

The use of thickeners like, e.g., aluminum monostearate, hydrogenated castor oil, carboxymethyl cellulose, or salts thereof can be suitable as well.

Emulsifiers may be present, if desired. The preferred emulsifiers used, apart from the emulsifiers known from the prior art, include polyoxyethylene derivatives of castor oil or polyoxyethylene alkylethers.

If the application form selected requires a foam-forming agent, it may be used any of those which are permitted under the drug licensing laws and known from the prior art, preferably polyoxyethylene sorbitan esters of various fatty acids (polysorbates).

Suitable propellant gases which may be used are all those which are licensed for use in the medical field and those which are known from the prior art, e.g., CO₂, N₂O, N₂, propane/butane mixtures, isobutane, chloropentafluoroethane (CClF₂-CF₃), and octafluorocyclobutane (C₄F₈).

It is a matter of course that all generally used additives known and accepted for pharmaceutical application may be present in the formulations of the present invention in the usual amounts.

Aqueous based formulations for the preparation of a veterinary medical composition will now be illustrated by the Examples. However, it is expressly pointed out that the Examples are intended solely as an illustration and should not be regarded as restricting the invention.

### EXAMPLES

In the following Examples 1 to 3 formulations according to the invention were prepared for intramammary use (in accordance with the requirements of Ph. Eur.) containing meloxicam or meloxicam salt in an aqueous or oily system. The formulations are listed in the following tables.

### Example 1

Formulation 1 of the invention was prepared in form of an injector solution.

Formulation 1

| ingredient | g/100 mL |
|---|---|
| Meloxicam | 0.500 |
| Meglumine | 0.3125 |
| Glycofurol | 10.000 |
| Poloxamer 188 | 5.000 |
| Ethanol | 15.000 |
| Sodium chloride | 0.600 |
| Glycine | 0.500 |
| Sodium hydroxide | q.s. to give pH 8.7 |
| Water for injection | to 100 mL |

### Example 2

Formulation 2 of the invention was prepared in form of an injector solution.

Formulation 2

| ingredient | g/100 mL |
|---|---|
| Meloxicam | 0.500 |
| Meglumine | 0.3125 |
| Glycofurol | 10.000 |
| Poloxamer 188 | 5.000 |
| Carboxymethylcellulose sodium | 5.000 |
| Ethanol | 15.000 |
| Sodium chloride | 0.600 |
| Glycine | 0.500 |
| Sodium hydroxide | q.s. to give pH 8.7 |
| Water for injection | to 100 mL |

### Example 3

Formulation 3 of the invention was prepared in form of an oily suspension.

Formulation 3

| ingredient | g/100 mL |
|---|---|
| Meloxicam | 2.000 |
| Aluminum monostearate | 2.000 |
| α-Tocopherol | 0.050 |
| Sesame oil | to 100 mL |

In the following Examples 4 to 8, formulations according to the invention were prepared for oral or parental use containing meloxicam or meloxicam salt. The formulations are listed in the following tables.

EXAMPLE 4
2% Meloxicam Solution

| Component | Amount (g/L) |
|---|---|
| Meloxicam | 20.0 |
| Meglumine | 14.0 |
| Macrogol 300¹ | 150.0 |
| Poloxamer 188² | 50.0 |
| Ethanol | 150.0 |
| Glycine | 5.0 |
| EDTA-Na | 1.0 |
| 1M HCl | q.s. to pH 8.8 |
| 1M NaOH | q.s. to pH 8.8 |
| Water for injections | to 1000 mL |

Legend:
¹obtainable from Brenntag, Plochingen, Germany; and
²obtainable from C. H. Erbsloeh, Krefeld, Germany

Method: 20 g of meloxicam is dissolved in 500 mL of an aqueous meglumine solution (14 g/500 mL) at 90° C. The other excipients are added one after another to the solution according to the recipe given above. A pH of 8.8 is then achieved using 1M hydrochloric acid and 1M sodium hydroxide solution. Water is added to the solution until a volume of 1 liter is obtained.

EXAMPLE 5
2% Meloxicam Solution

| Component | Amount (g/L) |
|---|---|
| Meloxicam | 20.0 |
| Meglumine | 12.5 |
| PEG 400 | 100.0 |
| Poloxamer | 50.0 |
| Ethanol | 150.0 |
| Glycine | 5.0 |
| EDTA-Na | 1.0 |
| 1M HCl | q.s. to pH 8.8 |
| 1M NaOH | q.s. to pH 8.8 |
| Water for injections | to 1000 mL |

Method: 20 g of meloxicam is dissolved in 500 mL of an aqueous meglumine solution (12.5 g/500 mL) at 90° C. The other excipients are added one after another to the solution according to the recipe given above. A pH of 8.8 is then achieved using 1M hydrochloric acid or 1M sodium hydroxide solution. Water is added to the solution until a volume of 1 liter is obtained.

EXAMPLE 6
2.5% Meloxicam Solution

| Component | Amount (g/L) |
|---|---|
| Meloxicam | 25.0 |
| Meglumine | 17.5 |
| PEG 300 | 150.0 |
| Poloxamer | 50.0 |
| Ethanol | 150.0 |
| Glycine | 5.0 |
| EDTA-Na | 1.0 |
| 1M HCl | q.s. to pH 8.8 |
| 1M NaOH | q.s. to pH 8.8 |
| Water for injections | to 1000 mL |

Method: 25 g of meloxicam is dissolved in 500 mL of an aqueous meglumine solution (17.5 g/500 mL) at 90° C. The other excipients are added one after another to the solution according to the recipe given above. A pH of 8.8 is then achieved using 1M hydrochloric acid or 1M sodium hydroxide solution. Water is added to the solution until a volume of 1 liter is obtained.

EXAMPLE 7
1.5% Meloxicam Solution

| Component | Amount (g/L) |
|---|---|
| Meloxicam | 15.0 |
| Meglumine | 10.5 |
| PEG 300 | 100.0 |
| Poloxamer | 50.0 |
| Ethanol | 150.0 |
| Glycine | 5.0 |
| EDTA-Na | 1.0 |
| 1M HCl | q.s. to pH 8.8 |
| 1M NaOH | q.s. to pH 8.8 |
| Water for injections | to 1000 mL |

Method: 15 g of meloxicam is dissolved in 500 mL of an aqueous meglumine solution (10.5 g/500 mL) at 90° C. The other excipients are added one after another to the solution according to the recipe given above. A pH of 8.8 is then achieved using 1M hydrochloric acid or 1M sodium hydroxide solution. Water is added to the solution until a volume of 1 liter is obtained.

EXAMPLE 8
2% Meloxicam Solution

| Component | Amount (g/L) |
|---|---|
| Meloxicam | 20.0 |
| Meglumine | 14.0 |
| PEG 300 | 150.0 |
| Poloxamer | 50.0 |
| p-Chloro-m-cresol | 2.0 |
| Glycine | 5.0 |
| EDTA-Na | 1.0 |
| 1M HCl | q.s. to pH 8.8 |
| 1M NaOH | q.s. to pH 8.8 |
| Water for injections | to 1000 mL |

Method: 20 g of meloxicam is dissolved in 500 mL of an aqueous meglumine solution (14 g/500 mL) at 90° C. The other excipients are added one after another to the solution according to the recipe given above. A pH of 8.8 is then achieved using 1M hydrochloric acid or 1M sodium hydroxide solution. Water is added to the solution until a volume of 1 liter is obtained.

The formulation used according to the invention is suitable for preparing a veterinary composition having analgesic effects, particularly for treating mild and moderate mastitis cases. It is suitable for treating mammals, particularly working animals or farm animals. The treatment may be given in conjunction with antibiotic therapy administered systematically and/or locally. It is possible to treat large farm animals with a meloxicam formulation suitable for treating farm animals up to 750 kg in weight. It is preferred that the pharmaceutical composition is used in form of a solution which is free from particles, particularly in case of parenteral administration.

Since one single dose may be sufficient, the dosage of the formulation according to the invention should correspond to 0.2 to 1.0 mg of active substance per kg of bodyweight, preferably 0.3 to 0.8 mg/kg of bodyweight, more preferably 0.4 to 0.7 mg/kg of bodyweight, particularly preferably 0.4 to 0.6 mg/kg of bodyweight, especially about 0.5 mg/kg of bodyweight.

The formulation according to the invention may be prepared using the methods of preparing formulations known from the literature. For example, the appropriate additives may be added to a meloxicam/meloxicam salt preparation.

The meloxicam containing formulation of the invention may be administered in the form of creams, ointments, lotions, gels, water-in-oil or oil-in-water emulsions, aerosol foams, solutions, or suspensions, for example, on the basis of water, ethanol, or a mixture thereof. Particularly preferred are any kind of injector and injection formulations, e.g., such as intracutaneous or subcutaneous needleless injection or injector formulations with a blunt needle for intramammary injection or ready to use syringes, or injection formulations for parenteral application, such as i.v. or i.m. injection. The preparation of pharmaceutical forms of this kind is well-known per se from the prior art.

Already known or licensed meloxicam formulations, such as solutions for injection available on the market, may be used. Due to the long duration of action of meloxicam in cattle, preferably a single treatment will provide a long lasting analgesic efficacy, which contributes to animal wellbeing. Therefore, a single treatment such as a single shot or single dose may provide a long lasting reduction of the hypersensitive states/inflammatory hyperalgesia, i.e., painful conditions related to mild and moderate mastitis cases. A single administration of the veterinary medical composition is preferably sufficient for the treatment of an inflammatory painful disease, in particularly reduction of local inflammatory signs in the affected quarter, i.e., reduction of swelling, redness, heat, and pain and restore normal functions (normal milk production combined with decrease of the somatic cell counts), and should be understood in that treatment with one dose of the formulation reduces the above mentioned local inflammatory signs and restores normal behavioral responses to pain stimuli fast, efficacious, and long lasting.

The requirements imposed on an active substance containing formulation includes inter alia small volumes or amounts to be administered, the possibility of weight-related dosage and maximum possible flexibility in the number of actuation processes per treatment unit. Accordingly, injection volumes of 50 μL per actuation, for example, are technically feasible. For this purpose, as described in DE 100 10123 A1, a sterile solution may be transferred under aseptic conditions into a sterile cartridge which is then inserted in the metering system.

The formulation used according to the present invention makes it possible for the animal keeper himself to administer the sterile formulation to the animal. The formulation of the present invention is preferably prepared for administration by parenteral or intramammary route. Therefore, the formulation may be administered systemically through the parenteral route, i.e., the active substance occurs in the blood of the animal or it may be administered locally through the intramammary route, i.e., the active substance is applied directly on or into the affected site (udder).

Preferably use is made of an injection formulation which is known per se by the skilled person. The injection formulation is preferably selected from the abovementioned aqueous system.

Preferably used are also injector formulations. An injector comprises means which allow to inject the formulation intramammary, i.e., through the streak canal into the udder, with the formulation being present in a casing, reservoir, phial, syringe, or tube or the like, which may be disposable and provided for a single-use, containing a delivery system such as a suitable opening, channel or a blunt needle. This form of intramammary application may achieve a good distribution in the target organ together with an increase in the activity. The injector formulation is preferably selected from the abovementioned oily or aqueous system.

The usual shelf-life of the formulation after opening is about several weeks or more at ambient (normal room) temperature. The shelf-life of the formulation in the sealed original packaging may be up to one or several months or more. The formulation was found to be stable even when subjected to the process of final sterilization.

The advantages of the present invention are manifold: A new method for the assessment of painful conditions in food producing animals has made it possible to treat cows suffering from mild to moderate mastitis cases. A clinical field study based on such method shows the analgesic efficacy of meloxicam and confirms results which indicate that a single treatment with meloxicam formulation achieves a long lasting analgesic efficacy. The analgesic effect is observed to occur very quickly. A single administration has a long lasting effect over several days resulting in a tremendous improvement of the wellbeing of the ill animals whereas a potent and rapid alleviation of pain is observed.

Therefore, the meloxicam formulation may be used for the treatment of mild and moderate mastitis cases. The treatment leads to an effective long lasting reduction of a hypersensitive state associated with inflammatory pain in mild and moderate mastitis cases, particularly in chronic states.

The other known NSAIDs which are only capable to be parenterally administered may not be used in (chronic) mild or moderate mastitis cases due to their either weak analgesic efficacy and/or due to the short duration of action of below 12 hours.

The experimental findings described hereinafter provide clear evidence of successful treatment of pain of moderate or mild mastitis in mammals by the use of the meloxicam formulation of the present invention.

The invention described will now be illustrated by the Examples which follow various other embodiments and will become apparent to the skilled person from the present specification. However, it is expressly pointed out that the Examples and description are intended solely as an illustration and should not be regarded as restricting the invention.

### STUDY EXAMPLES

Study Example 1 describes the method validation for the mechanical device measuring hypersensitivity in cows. This study was undertaken to assess the use of a range of clinical and laboratory parameters in assessing pain in dairy cows with mild and moderate clinical mastitis.

Study Example 2 describes a field study which has been conducted to show the long lasting analgesic as well as anti-inflammatory efficacy of a 2% meloxicam formulation in cows with mild and moderate mastitis cases.

### Study Example 1

The Method Validation

Pain in dairy cows with mild and moderate clinical mastitis was assessed using the characterization of peripheral inflammatory mediators in the regulation of inflammatory hyperalgesia in dairy cows.

Dairy cows were examined clinically and milk samples were collected for bacteriological culture and quality analyses, on the day of diagnosis.

The distance between the hocks was measured as a proxy indicator of altered cow stance. Response thresholds to mechanical stimuli were measured on each hind limb using a modification of the method described by Nolan and others (A. Nolan, A. Livingston, R. Morris, and A. Waterman, Techniques for comparison of thermal and mechanical nociceptive stimuli in the sheep, J. Pharmacol. Methods 1987, 17, pp. 39-49).

Kruskal-Wallis and one-way ANOVA tests were used to compare parameters from mild and moderate cases of mastitis and normal cows.

Overall, 117 lactating cows with clinical mastitis (n=61 mild; n=56 moderate) and 45 normal cows were studied. The bacteriological results showed that Escherichia coli was isolated from 28%, and Streptococcus uberis from 39% of moderate cases; while in mild cases, E. coli and S. uberis, accounted for 16% and 18% of cases, respectively. The hock-hock distance and mechanical threshold difference were lower in normal cows than in cows with mastitis (both mild and moderate cases) (p<0.001). The heart rates, respiratory rates and rectal temperatures of cows with moderate mastitis were higher (p<0.001) than cows with mild mastitis, and normal animals. The individual quarter somatic cell count (IQSCC) and protein content of the milk of normal animals were lower compared to cows with mastitis (both mild and moderate cases; p<0.001) and the lactose content of milk was higher in normal animals compared to cases with mastitis (both mild and moderate; p<0.001).

The results suggest that cows with mild and moderate mastitis exhibit mechanical hyperalgesia, indicating altered pain processing as a consequence of the inflammatory disease. These results indicate that techniques can be used to monitor pain indirectly in cattle with mild and moderate mastitis. Furthermore, the response to analgesic treatments can be assessed quantitatively.

### Study Example 2

Clinical Field Study

Preliminary results on the effects of meloxicam (Metacam®)) on hypersensitivity in dairy cows with clinical mastitis

Recognition, alleviation and control of pain and stress are central to ensuring good welfare in food producing animals. Over 100 dairy cows with clinical mild or moderate mastitis were studied to assess pain associated with clinical mastitis. Mastitis therapy was given according to routine veterinary practice, with intramammary antibiotic drugs. A preparation without corticosteroid was selected, cefquinome (Cephaguard LC Intramammary, Intervet UK Limited, Milton Keynes), this was infused every 12 hours for three treatments for each case of mastitis. Cows with clinical mastitis were allocated randomly to one of 3 groups:
Group 1: antibiotics only;
Group 2: antibiotics and one dose of meloxicam (sold under the Metacam® trademark by Boehringer Ingelheim);
Group 3: antibiotics and three doses of meloxicam on day of diagnosis, day 0, and on days 3 and 6.
Healthy animals were recruited as controls.

All cows were examined clinically on 6-8 occasions over a 45 day period. Response thresholds to mechanical stimuli were measured on each hind limb. General linear model in Mintab Statistical Software (Minitab Inc.) and multi-level modeling in MLwiN (multilevel factor analysis model for data evaluation) were used to consider the time effect and treatment effect.

Treatment had a significant effect on threshold responses, with cows that received antibiotics alone (Group 1) showing greater differences compared to cows that received either one (Group 2) or three (Group 3) doses of meloxicam (P<0.001). There was no significant difference in cows that received one, compared to three doses of meloxicam.

There was a significant effect of time on threshold responses for cows with mild cases of mastitis only on day 45 compared to the day of diagnosis (P<0.05).

In conclusion, mechanical hyperalgesia is present in animals with mastitis of both mild and moderate severity. Treatment with one or three doses of meloxicam was shown to restore normal threshold responses to mechanical stimuli.

These results indicate that meloxicam is beneficial in analgesic therapy of mild and moderate clinical mastitis in dairy cows. Meloxicam treatment restores normal behavioral responses to pain stimuli.

Conclusion

Single treatment with meloxicam 2% solution for injection showed long lasting analgesic efficacy in lactating cows suffering from mild and moderate chronic mastitis.

1. A method of treating an inflammatory painful disease in a non-human mammal, the method comprising administering to the non-human mammal in need thereof a pharmaceutical formulation comprising an effective amount of meloxicam or a pharmacologically acceptable meloxicam salt of an organic or inorganic base and a vehicle.

2. The method according to claim 1, wherein the non-human mammal is a bovine.

3. The method according to claim 1, wherein the non-human mammal is a cow.

4. The method according to claim 3, wherein the inflammatory painful disease is mild or moderate mastitis.

5. The method according to one of claims 1 to 4, wherein the concentration of the meloxicam or meloxicam salt in a pharmaceutical formulation is 10-30 mg/mL.

6. The method according to one of claims 1 to 4, wherein the method is accomplished by a single administration of the pharmaceutical formulation.

7. The method according to one of claims 1 to 4, wherein the dosage of corresponds to a dosage range of from 0.2 to 1.0 mg of active substance/kg of bodyweight.

8. The method according to one of claims 1 to 4, wherein the vehicle is water and/or oil.

9. The method according to one of claims 1 to 4, wherein the pharmaceutical formulation further comprises one or more suitable additives.

10. The method according to claim 9, wherein the additives are buffers, solubilizers, gelling agents, viscosity enhancers, preservatives, oils, antioxidants, emulsifiers, foam forming agents, isotonic agents, a propellant gas, or thickeners.

11. The method according to one of claims 1 to 4, wherein the pharmaceutical formulation consists essentially of a meloxicam salt, water, and one or more additives selected from buffers, solubilizers, preservatives, and thickeners.

12. The method according to one of claims 1 to 4, wherein the pharmaceutical formulation consists essentially of meloxicam, meglumine, water, a polyethyleneglycol, a polyethylene-polyoxypropylene copolymer, ethanol, glycine, and optionally sodium hydroxide or hydrochloric acid, and disodium EDTA.

13. The method according to one of claims 1 to 4, wherein the pharmaceutical formulation consists essentially of meloxicam, one or more oils, optionally one or more antioxidants, and optionally one or more thickeners.

14. The method according to one of claims 1 to 4, wherein the pharmaceutical formulation is administered parenterally or intramammarily.
